(19) United States
(12) Patent Application Publication
(10) Pub. No.: US 2026/0335118 A1
(43) Pub. Date: Oct. 29, 2026

(54) MODULAR ORTHOSIS SYSTEM

(71) Applicant: Messiah University, Mechanicsburg, PA (US)

(72) Inventors: Ryan James FARRIS, Dillsburg, PA (US); Laurel Elsie PETTITT, Salisbury, MD (US); Sarah GILBERT, Shoemakersville, PA (US)

(73) Assignee: Messiah University, Mechanicsburg, PA (US)

(21) Appl. No.: 19/659,001

(22) Filed: Apr. 27, 2026

Related U.S. Application Data
(60) Provisional application No. 63/796,021, filed on Apr. 28, 2025.

(51) Int. Cl. A61F 5/058

(52) U.S. Cl. CPC A61F 5/0585

(57) ABSTRACT

A modular orthosis system includes a thigh yoke removably securable to a thigh shell, which engages and removably secures to a wearer’s leg above the knee. A shank yoke is removably securable to a shank shell, which engages and removably secures to a wearer’s leg below the knee. The thigh and shank yokes are removably and rotatably connected via a pair of thigh yoke joints, each thigh yoke joint being removably securable to the thigh yoke, and a pair of shank yoke joints, each shank yoke joint being removably securable to the shank yoke. Different sizes of shells and yokes may be utilized to provide an orthosis with a customizable fit. The shells and yokes can be disassembled and stored in a stacked or nested configuration for ease of shipment and storage. The system allows for easy incorporation of a wide range of commercially available and custom knee joints.

### CROSS REFERENCE TO RELATED APPLICATIONS

This application claims the benefit of U.S. Provisional Patent Application No. 63/796,021, filed Apr. 28, 2025, which is hereby incorporated by reference in its entirety as if fully set forth.

### BACKGROUND OF THE INVENTION

Knee orthoses (KOs) and knee-ankle-foot orthoses (KAFOs) are commonly prescribed to provide gait stability to individuals with lower extremity mobility impairments. Conventionally, the orthoses are custom fabricated to fit a single individual, providing an optimal fit for the wearer. The custom fabrication process often takes weeks or months starting with an evaluation visit in a clinic, followed by a leg casting or scanning process, then off-site fabrication of the prescribed orthosis design, and a final visit to receive and be fitted in the new orthosis.

The extensive and expensive process of custom fabrication has largely precluded the use of orthoses in a therapeutic rehabilitation setting, despite the fact that many patients receiving therapy might benefit from their use and may not have one of their own. This issue has failed to be addressed in part for two reasons. First, the added benefit of using traditional orthoses in gait therapy may not be significant in many cases. Second, in developed countries, traditional leg orthoses are affordable and covered by insurance for most who would need them, so individuals often do have their own custom orthosis which they would bring to gait therapy sessions.

The need for a clinical-use lower extremity orthosis which can accommodate a wide range of patient anthropometrics has grown in recent years. Advancements in orthosis-enabling technologies such as microprocessor-controlled knee joints with electromechanical actuation make the use of advanced orthoses in rehabilitation therapy more desirable while simultaneously making the cost point of these orthoses less attainable for patients to own on an individual basis. Some examples of orthoses that utilize microprocessor-controlled knee joints are disclosed in U.S. Patent No. 10,390,973. Such orthosis technologies have been demonstrated to support higher quality gait therapy sessions (e.g., more consistent step patterns with more controllable support) while delivering higher dosage sessions (i.e. more steps) with reduced clinician fatigue.

Some manufacturers have tried to address this gap by creating full lower extremity “exoskeletons” which generally comprise rigid limb segments housing motors, gearing, sensors, and batteries, and which are strapped to the patient’s corresponding limb segments. These systems tend to be heavy, bulky, and have limited ability to provide a close custom fit to the wearer. Many of these exoskeletons attempt to provide adjustability (e.g., through telescoping limb segments) to accommodate a wide range of patient sizes. This addition of adjustment mechanisms unfortunately compounds the issue of bulk and weight.

While some have attempted to achieve a desirable orthosis fit by increasing flexibility to accommodate a wide range of body types, this sacrifices structural rigidity, jeopardizing the support, control, and confidence of the wearer. Therefore, it is desirable to provide an orthosis with a modular design, which provides a near-custom fit while retaining a critical level of rigidity for secure support to the wearer.

The present invention addresses the previously described need by providing a modular orthosis system that includes combination of rigid, pivoting thigh and shank frames with load-centralizing connectors for the attachment of flexible (i.e., less rigid) body-interface shells. In this way, the modular orthosis system is rigid enough to provide support and transmit high torque while being flexible enough to provide comfort against soft tissue and fit a wide range of body types.

The present invention also addresses the previously described need through a modular “kit” of components which can be combined to fit any patient in a clinical rehab setting within a certain height and weight range. The present invention is adapted to support the use of an electromechanical stance control knee joint, but could also accommodate a wide range of knee joints ranging from simple passive locking joints to spring loaded joints with passive dampers, to fully powered intelligent knee joints that are currently under development.

Further, the present invention includes a series of nesting components which can be stored or transported in a small bag, which presents a distinct advantage over devices which are non-modular, and therefore must remain fully assembled, making transport and storage more difficult.

Unlike the expensive and often intricate adjustment mechanism found in some exoskeleton products, the present invention has been intentionally designed for ease of manufacture, with the goal of minimizing fabrication complexity and reducing the cost of goods. As a result, the present invention may be produced at a cost which is two orders of magnitude lower than the current price point of commercially available exoskeletons, even when manufactured in low quantities.

An additional advantage provided by the present invention is the ability to position supporting thigh and shank “shells” on either the anterior or posterior aspect of the leg. This provides flexibility to the clinician to assemble the ideal configuration for each patient, based on their individual presentation as well as their specific body shape. In other exoskeletons and leg orthoses, the positioning of the leg shells or “cuff” is fixed.

### SUMMARY OF THE INVENTION

In one embodiment of the present invention, the modular orthosis system, includes a thigh yoke comprising a connector portion configured to removably secure to a thigh shell, the thigh shell adapted to engage and removably secure to a wearer’s leg above the knee. A shank yoke comprises a connector portion configured to be removably securable to a shank shell, the shank shell adapted to engage and removably secure to a wearer’s leg below the knee. The system includes a pair of thigh yoke joints, each thigh yoke joint being removably securable to the thigh yoke, and a pair of shank yoke joints, each shank yoke joint being removably securable to the shank yoke. Each thigh yoke joint is configured to be removably and rotatably connected to one of the shank yoke joints. Different sizes of shells and yokes may be utilized to provide an orthosis with a customizable fit. The shells and yokes can be disassembled and stored in a stacked or nested configuration for ease of shipment and storage.

One object of the present invention is to provide a modular orthosis system that is usable with different types of joint connectors, including, for example, electromechanical joint connectors.

Another object of the present invention is to provide a modular orthosis system that may include an optional anti-slip element that provides additional support and helps prevent the modular orthosis system from slipping downwardly along the wearer’s leg.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

The present invention will hereinafter be described in conjunction with the appended drawing figures wherein like numerals denote like elements.

FIG. 1 is a front perspective view of a first embodiment of the modular orthosis system in an assembled configuration.

FIG. 2 is a rear perspective view of a first embodiment of the modular orthosis system in an assembled configuration.

FIG. 3A is a rear elevation view of a first embodiment of the modular orthosis system in a first assembled configuration with small-sized shell and yoke components.

FIG. 3B is a rear elevation view of a first embodiment of the modular orthosis system in a second assembled configuration with medium-sized shell and yoke components.

FIG. 3C is a rear elevation view of a first embodiment of the modular orthosis system in a third assembled configuration with large-sized shell and yoke components.

FIG. 4A is a rear perspective view of a first embodiment of the modular orthosis system in a first assembled configuration with small-sized shell and yoke components.

FIG. 4B is a rear perspective view of a first embodiment of the modular orthosis system in a second assembled configuration with medium-sized shell and yoke components.

FIG. 4C is a rear perspective view of a first embodiment of the modular orthosis system in a third assembled configuration with large-sized shell and yoke components.

FIG. 5A is a perspective view of a first embodiment of the modular orthosis system in a fourth assembled configuration, with both the shank shell and thigh shell in an anterior orientation.

FIG. 5B is a perspective view of a first embodiment of the modular orthosis system in a fifth assembled configuration, with the shank shell in a posterior orientation and the thigh shell in an anterior orientation.

FIG. 5C is a perspective view of a first embodiment of the modular orthosis system in a sixth assembled configuration, with the shank shell in an anterior orientation and thigh shell in a posterior orientation.

FIG. 5D is a perspective view of a first embodiment of the modular orthosis system in a seventh assembled configuration, with both the shank shell and thigh shell in a posterior orientation.

FIG. 6 is a perspective view of a first embodiment of the modular orthosis system showing different sizes of yokes and leg shells in a disassembled and stacked configuration.

FIG. 7 is a perspective view of a first embodiment of the modular orthosis system showing different sizes of yokes and leg shells in a partially assembled and stacked configuration.

FIG. 8 is a rear elevation view of the thigh yoke and thigh shell of a second embodiment of the modular orthosis system in a partially assembled configuration.

FIG. 9 is a rear perspective view of a third embodiment of the modular orthosis system in an assembled configuration.

FIG. 10 is a rear elevation view of a third embodiment of the modular orthosis system in an assembled configuration.

FIG. 11 is a side elevation view of a third embodiment of the modular orthosis system in an assembled configuration.

FIG. 12 is a diagram showing a simulated strain response plot during a wearer’s gait in an embodiment of the modular orthosis system that includes a strain gauge.

FIG. 13 is a perspective view showing an anti-slip element of a fourth embodiment of the modular orthosis system.

FIG. 14 is a rear elevation view of a fourth embodiment of the modular orthosis system with an attached anti-slip element.

FIG. 15 is a rear elevation view of a fourth embodiment of the modular orthosis system with an attached anti-slip element and securement strap.

FIG. 16 is a perspective view of a fourth embodiment of the modular orthosis system secured to a wearer’s leg.

### DETAILED DESCRIPTION OF THE INVENTION

The ensuing detailed description provides preferred exemplary embodiments only, and is not intended to limit the scope, applicability, or configuration of the invention. Rather, the ensuing detailed description of the preferred exemplary embodiments will provide those skilled in the art with an enabling description for implementing the preferred exemplary embodiments of the invention. It should be understood that various changes may be made in the function and arrangement of elements without departing from the spirit and scope of the invention, as set forth in the appended claims.

As used herein, the term “flexural modulus” refers to a measure of a material’s resistance to elastic deformation under bending and is defined as the ratio of stress to strain in the linear (elastic) region of a stress–strain curve obtained during flexural loading. Flexural modulus characterizes the stiffness of a material when subjected to bending forces and may be expressed in units of pressure, such as pascals (Pa) or pounds per square inch (psi). Unless expressly stated otherwise, references to flexural modulus are not limited to a particular test method, specimen geometry, loading rate, or environmental condition.

As used herein, the term “shape,” when referring to a thigh yoke, shank yoke, thigh shell, or shank shell, means the three‑dimensional geometric form and contour of the yoke or shell, including, without limitation, its curvature distribution, cross‑sectional profile, wrap angle around the limb, asymmetry (e.g., medial/lateral, anterior/posterior), offsets, flares, surface reliefs, depressions, cut‑outs or windows, perforation or slot patterns, bosses, ribs, fillets, chamfers, and edge geometries, as well as the spatial relationship among these features. Unless expressly stated otherwise, “shape” refers to the as‑manufactured geometry of the yoke or shell, prior to any patient‑specific adjustment or in‑use deformation.

As used herein, the term “size,” when referring to a thigh yoke, shank yoke, thigh shell, or shank shell, refers to the absolute scale or overall dimensions of the yoke or shell.

To aid in describing the invention, directional terms are used in the specification and claims to describe portions of the present invention (e.g., upper, lower, left, right, upward, downward, etc.). These directional definitions are merely intended to assist in describing and claiming the invention and are not intended to limit the invention in any way. In addition, reference numerals that are introduced in the specification in association with a drawing figure may be repeated in one or more subsequent figures without additional description in the specification in order to provide context for other features.

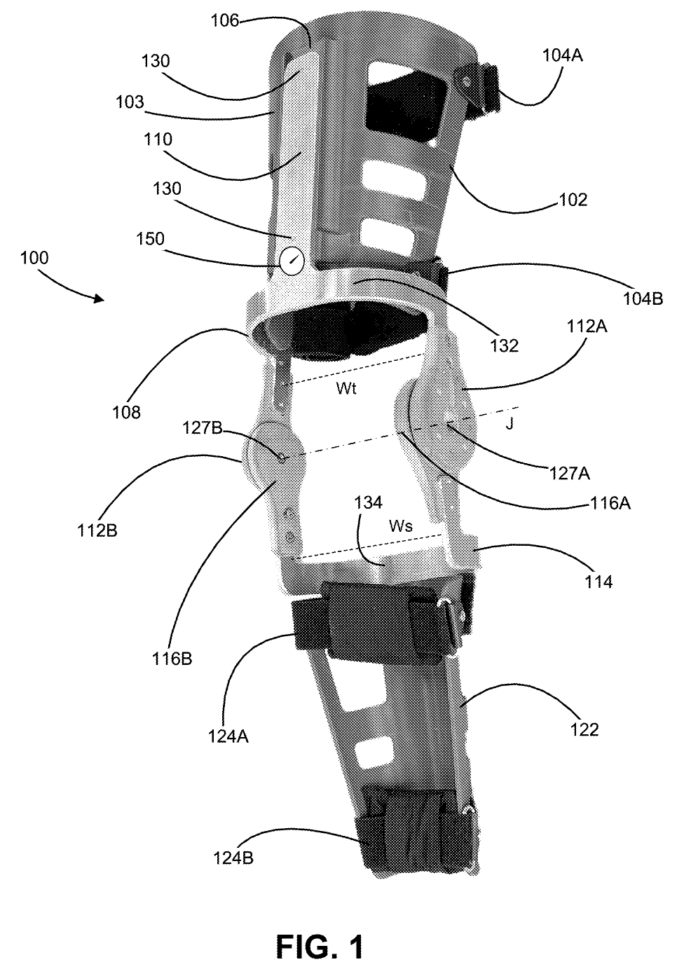

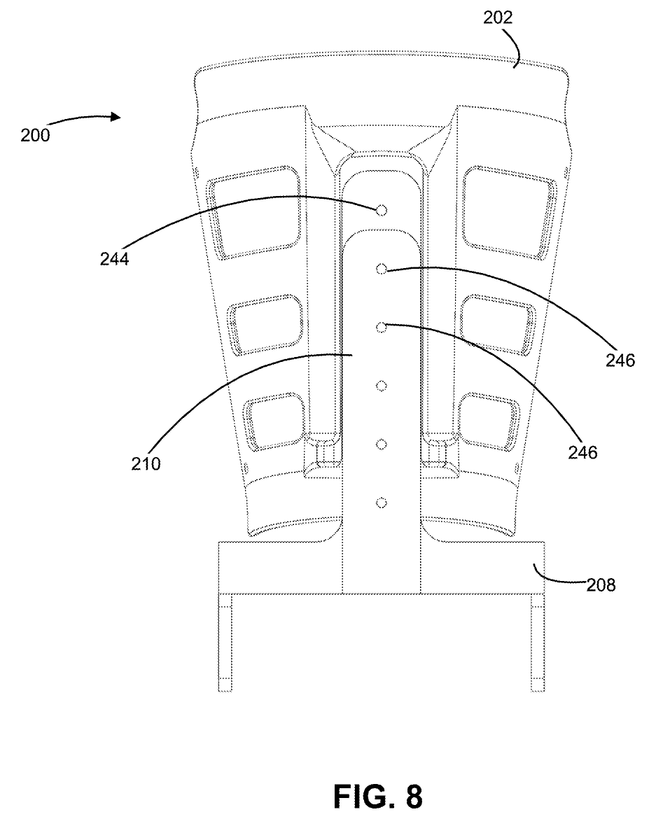

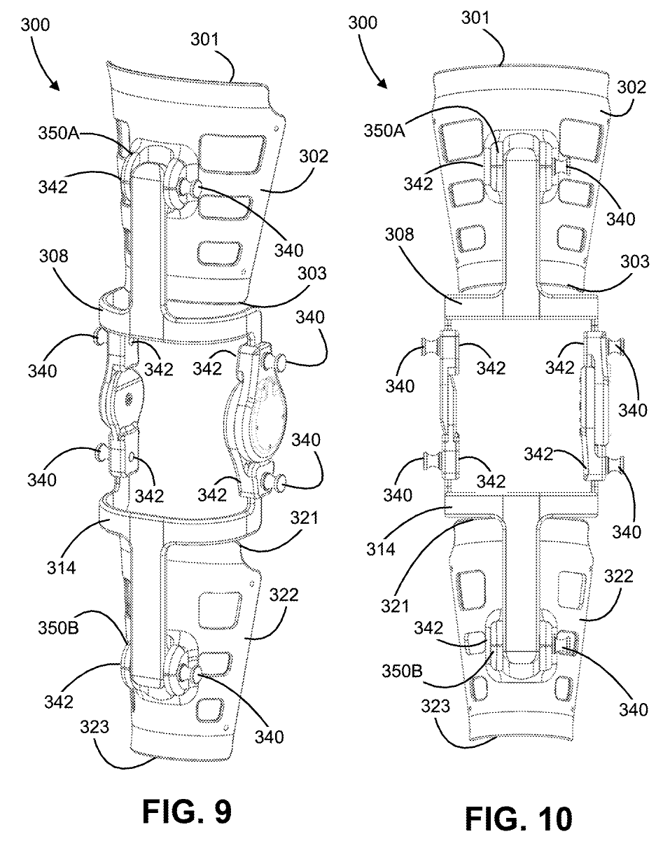

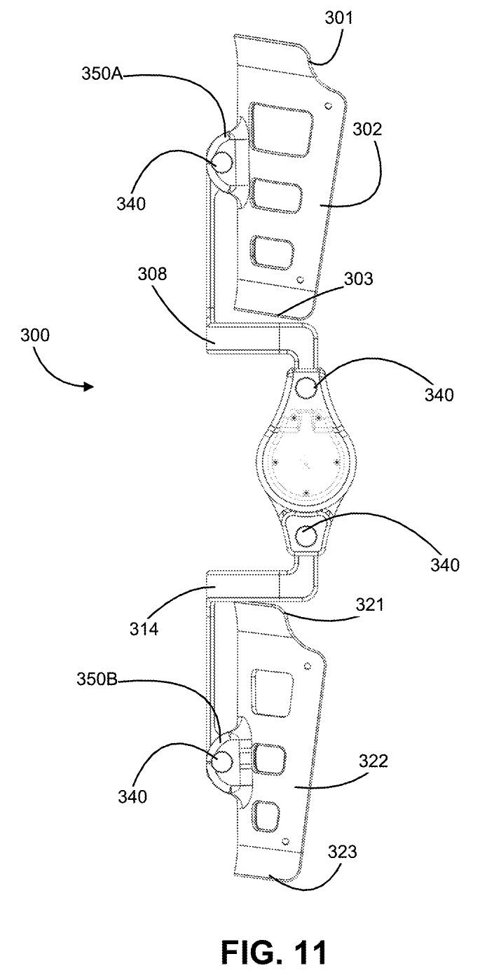

In subsequent embodiments of the present invention shown in the drawings, elements shared with the first embodiment are represented by reference numerals increased by factors of 100. For example, the modular orthosis system 100 in FIGS. 1-7 corresponds to the modular orthosis system 200 in FIG. 8, and to the modular orthosis system 300 in FIGS. 9-11. In the interest of clarity, some features of these embodiments that are shared with the first embodiment are numbered in FIGS. 8 and 9-11 but are not repeated in the specification.

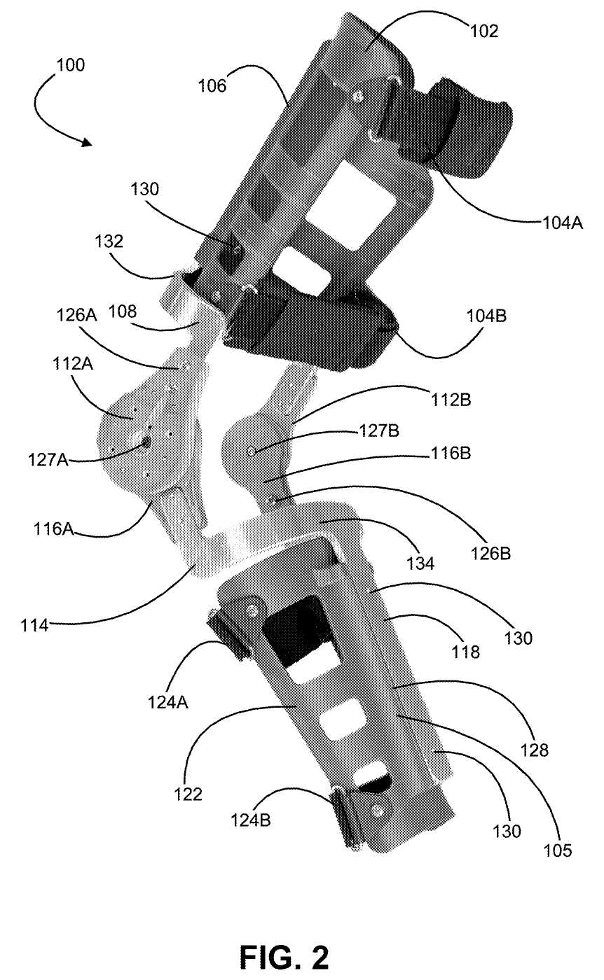

Referring to FIGS. 1 and 2, a first embodiment of the modular orthosis system 100 includes two yoke elements, namely, a thigh yoke 108 and a shank yoke 114. The thigh yoke 108 and shank yoke 114 are each composed of a yoke material, which may be a metallic material such as aluminum in an exemplary embodiment, but may be any other suitable material. The thigh yoke 108 includes a first thigh yoke joint connector 112A and a second thigh yoke joint connector 112B, which are connected via a thigh yoke link 132, which is U-shaped in the illustrated embodiment. The thigh yoke joint connectors 112A, 112B define a width Wt of the thigh yoke 108 (visible in FIG. 1) extending between the first thigh yoke joint connector 112A and the second thigh yoke joint connector 112B. Likewise, the shank yoke 114 includes a first shank yoke joint connector 116A and a second shank yoke joint connector 116B, which are connected via a shank yoke link 134, which is also U-shaped in the illustrated embodiment. The shank yoke joint connectors 116A, 116B define a width Ws of the shank yoke 114 (visible in FIG. 1) extending between the first shank yoke joint connector 116A and the second shank yoke joint connector 116B.

The thigh yoke 108 includes a thigh shell connector 110 (visible in FIG. 1) that is rigidly connected to and extends upwardly from the thigh yoke link 132. The thigh shell connector 110 is located between the first thigh yoke joint connector 112A and the second thigh yoke joint connector 112B. Similarly, the shank yoke 114 includes a shank shell connector 118 (visible in FIG. 2) that is rigidly connected to and extends downwardly from the shank yoke link 134. The shank shell connector 118 is located between the first shank yoke joint connector 116A and the second shank yoke joint connector 116B. In an exemplary embodiment, the thigh shell connector 110 is centered on the thigh yoke link 132 between the first thigh yoke joint connector 112A and the second thigh yoke joint connector 112B, and the shank shell connector 118 is centered on the shank yoke link 134 between the first shank yoke joint connector 116A and the second shank yoke joint connector 116B. In another exemplary embodiment, the thigh shell connector 110 is located within a middle 30% of the thigh yoke width Wt, and the shank shell connector 118 is located within a middle 30% of the shank yoke width Ws.

A thigh shell 102 is adapted to engage a human thigh (i.e., a portion of either of the wearer’s legs located between the knee and the hip), and is further adapted to be rigidly connected to the thigh shell connector 110. A shank shell 122 is adapted to engage a human shank (i.e., a portion of either of the wearer’s legs located between the knee and the ankle) and is further adapted to be rigidly connected to the shank shell connector 118. In a preferred embodiment, the thigh shell 102 is removably attachable to the thigh shell connector 110, and the shank shell 122 is removably attachable to the shank shell connector 118.

The thigh shell 102 and the shank shell 122 are each composed of a shell material, which may be a polymeric material, for example, but may be any other suitable material. In an exemplary embodiment, the yoke material of the thigh and shank yokes 108, 114 has a yoke flexural modulus and the shell material of the thigh and shank shells 102, 122 has a shank flexural modulus that is less than the yoke flexural modulus. In other words, the thigh and shank shells 102, 122 are preferably more flexible and less rigid than the thigh and shank yokes 108, 114.

In the illustrated embodiment, the thigh shell 102 includes thigh straps 104A, 104B, and the shank shell 102, 122 includes shank straps 124A, 124B. The thigh straps 104A, 104B allow the thigh shell 102 to be adjustably secured to a human thigh, while the shank straps 124A, 124 allow the shank shell 122 to be adjustably secured to a human shank. The thigh and shank shells 102, 122 are designed to provide flexibility, supporting a secure fit around a variety of thigh and shank sizes once the thigh straps 104A, 104B and the shank straps 124A, 124B are tightened.

When the modular orthosis system 100 is in an assembled configuration, the first thigh yoke joint connector 112A and the first shank yoke joint connector 116A are each connected to a first pivoting joint 127A, and the second thigh yoke connector 112B and the second shank yoke joint connector 116B are each connected to a second pivoting joint 127B, such that the thigh yoke 108 and shank yoke 114 are joined on both the medial and lateral sides of the wearer’s leg. The pivoting joints 127A, 127B permit the thigh yoke 108 and the shank yoke 114 to pivot about a joint axis J (visible in FIG. 1).

Preferably, the first thigh yoke joint connector 112A and the first shank yoke joint connector 116A are each removably connected to the first pivoting joint 127A, and the second thigh yoke joint connector 112B and the second shank yoke joint connector 116B are each removably connected to the second pivoting joint 127B, which allows the modular orthosis system 100 to be easily disassembled for more compact and efficient storage and shipping. The thigh yoke joint connectors 112A, 112B, shank yoke joint connectors 116A, 116B, and first and second pivoting joints 126A, 126B may comprise any combination of electromechanical stance control knee joints, simple passive locking joints, spring loaded joints with passive dampers, or fully powered intelligent knee joints. Because the thigh and shank shells 102, 122 are centrally connected to the thigh and shank yokes 108, 114 via the thigh shell connector 110 and shank yoke connector 116, out-of-plane twisting moments are unlikely to be transmitted to the pivoting joints 127A, 127B. The two points of connection between the thigh and shank yokes 108, 114 creates a rigid structural frame around the wearer’s knee, which serves to isolate loading on the pivoting joints 127A, 127B to torsional loads around the axis of rotation. This is desirable because many commercially available knee joints are designed to provide resistive torque around its axis of rotation but are not designed to handle out-of-plane twisting.

In the illustrated embodiment, the thigh yoke shell connector 110 engages a thigh shell slot 106 (visible in FIG. 1) located on an exterior side 103 of the thigh shell 102, and the shank yoke shell connector 118 engages a slot 128 (visible in FIG. 2) located on an exterior side 105 of the shank shell 122. In the illustrated embodiment, the thigh and shank shell connectors 110, 118 are removably securable to thigh and shank shells 102, 122, respectively, via fasteners 130 that extend through apertures in the thigh and shank shell connectors 110, 118 and the thigh and shank shells 102, 122. The fasteners 130 may be threaded fasteners or any other suitable fastener. In the illustrated embodiment, the fasteners 130 include identical threaded Allen-head fasteners, although any other suitable fasteners may be utilized. In the illustrated embodiment, the fasteners 130 may be actuatable via an Allen key or a handheld electric driver, either of which may be provided with a kit that includes the modular orthosis system 100, in order to permit rapid assembly and disassembly in a clinical setting when the modular orthosis system 100 is being used to support gait therapy for patients.

In the illustrated embodiment, as shown schematically in FIG. 1, the modular orthosis system 100 may also include one or more strain gauges 150. An additional advantage provided by the present invention is the centralization and isolation of loads through the thigh shell connector 110 and the shank shell connector 118. The strain gauge 150 is adapted to conveniently and accurately measure and display brace loads in real time, specifically joint torque between the thigh yoke joint connectors 112A, 112B, the shank yoke joint connectors 116A, 116B, and the first and second pivoting joints 126A, 126B (which, collectively, form a knee joint). This provides critical information for accurate knee joint control in a modular orthosis system 100 that includes electromechanical joint brakes or actuators. Other known orthoses that utilize electromechanical joint brakes or actuators commonly rely on underfoot sensors which detect ground reaction forces to inform a controller when to actuate the joint brakes or actuators in order to lock the knee joint, unlock the knee joint, or provide powered movement to the knee joint. These types of sensors suffer from reliability challenges associated with in-shoe or under-shoe placement and create usability challenges associated with wires routed across the knee and ankle joints and into the shoe. The present invention provides a more user-friendly and reliable alternative sensing option which directly measures the load being supported by a locked knee joint, providing ideal feedback to a control system which is required to unlock the knee joint at precisely the right moment in the wearer’s gait cycle. In the illustrated embodiment, the strain gauge 150 is located on the thigh shell connector 110 of the thigh yoke 108, but the strain gauge 150 may be located elsewhere in other embodiments, such as elsewhere on the thigh yoke 108, on the shank shell connector 118 of the shank yoke 114, or elsewhere on the shank yoke 114, for example.

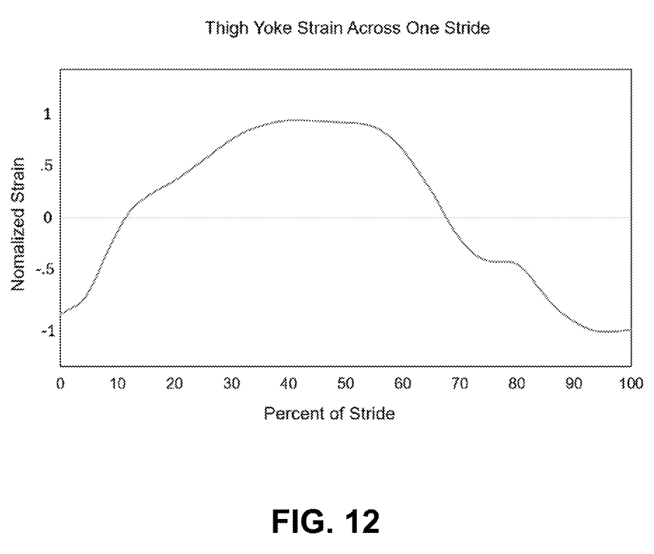

Referring to FIG. 12, the strain gauge 150 may be adapted to measure strain of the thigh yoke 108 across a wearer’s stride. In the graph shown in FIG. 12, positive normalized strain indicates elongation of the strain gauge 150 due to surface strain of the thigh yoke 108 while experiencing knee flexion torque, and zero percent of stride corresponds to heel strike of the braced leg. In other embodiments, the strain gauge 150 may be adapted to measure strain of the shank yoke 118 across one stride in lieu of or in addition to the strain of the thigh yoke 108.

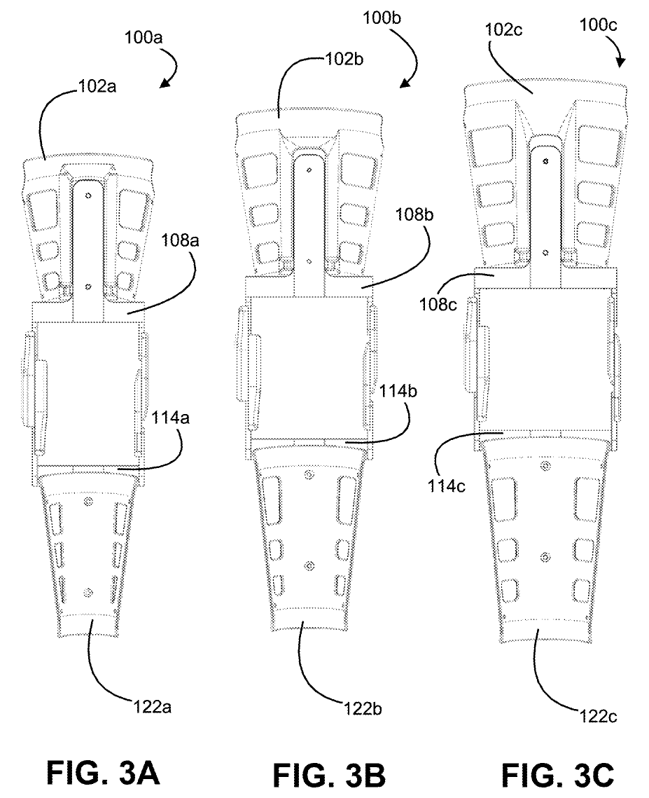

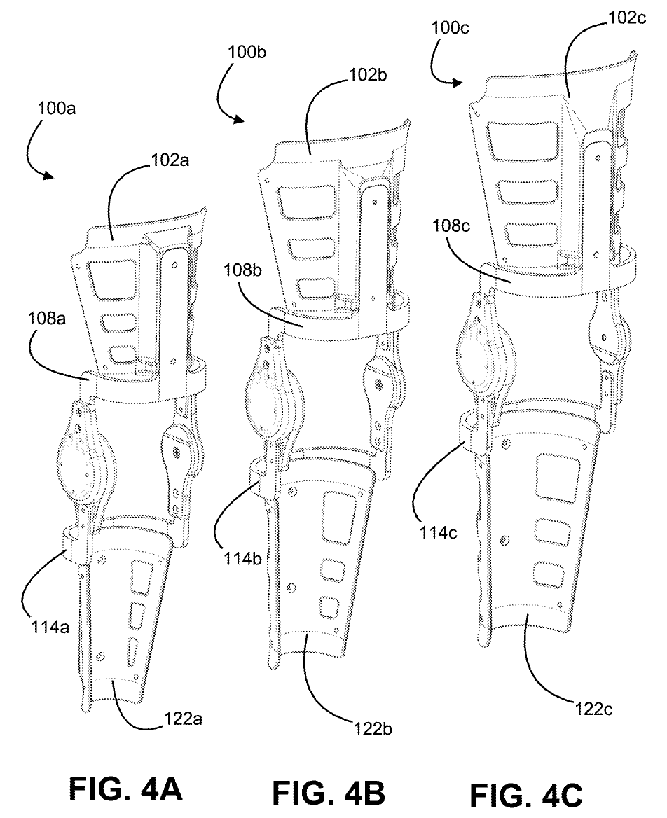

Referring to FIGS. 3A-3C and 4A-4C, the components of the modular orthosis system 100a-c may be provided in a range of shapes and sizes. FIGS. 3A and 4A show a modular orthosis system 100a assembled with relatively small-sized components, namely, a small thigh yoke 108a, a small thigh shell 102a, a small shank yoke 114a, and a small shank shell 122a. FIGS. 3B and 4B show a modular orthosis system 100b assembled with relatively medium-sized components, namely, a medium thigh yoke 108b, a medium thigh shell 102b, a medium shank yoke 114b, and a medium shank shell 122b. and FIGS. 3C and 4C show a modular orthosis system 100c assembled with relatively large-sized components, namely, a large thigh yoke 108c, a large thigh shell 102c, a large shank yoke 114c, and a large shank shell 122c. In other words, the small, medium, and large yokes and shells may each have a shape and size that differs from one another. More shapes and sizes may be developed as desired to cover a wider range of patients. Additionally, these components may be assembled in any combination (e.g. an assembled configuration of the system may include a medium thigh yoke 108b and medium thigh shell 102b with a small shank yoke 114a and small shank shell 122a, or may include a small thigh yoke 108a and small thigh shell 102a with a large shank yoke 114c and large shank shell 122c, etc.) to accommodate different leg shapes and sizes. In an exemplary embodiment, the system is adapted to accommodate a range of patient sizes from approximately a 15th percentile adult female up to an 85th percentile adult male.

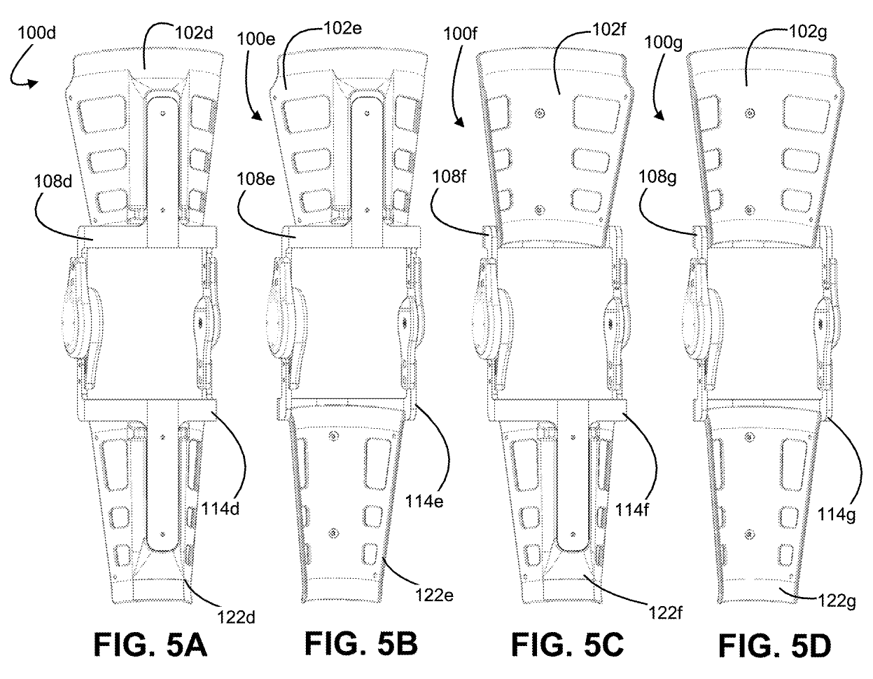

Referring to FIGS. 5A-5D, there are shown different assembled configurations of the first embodiment of the modular orthosis system. The modular orthosis system 100d-g permits flexible anteroposterior positioning of the thigh and shank shells 102d-g, 122d-g to provide optimal fit, maximal comfort, and ease of donning for the widest range of patients based on their own physiology and condition. For example, FIG. 5A shows the modular orthosis system 100d in an assembled configuration with both the shank shell 102d and thigh shell 122d in an anterior orientation. FIG. 5B shows the modular orthosis system 100e in an assembled configuration with the shank shell 122e in a posterior orientation and the thigh shell 102e in an anterior orientation. FIG. 5C shows the modular orthosis system 100f in an assembled configuration with the shank shell 122f in an anterior orientation and thigh shell 102f in a posterior orientation. FIG. 5D shows the modular orthosis system in an assembled configuration with both the shank shell 122g and thigh shell 102g in a posterior orientation.

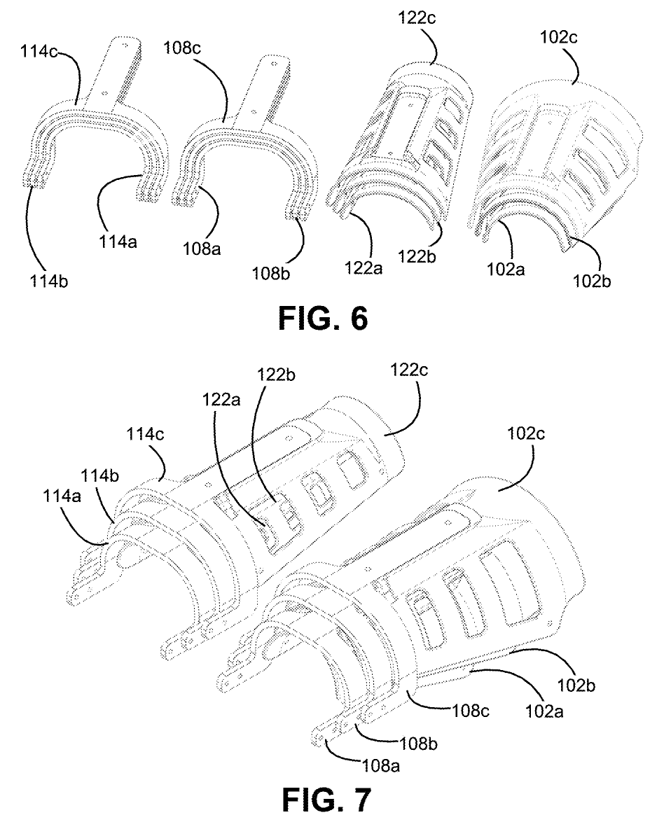

Referring to FIGS. 6 and 7, for ease of transport and storage, components of the system may be nested when disassembled, as shown in FIGS. 6 and 7. For example, as shown in FIG. 6, when the system is fully disassembled, small, medium, and large shank yokes 114a-c, small, medium, and large thigh yokes 108a-c, small, medium, and large shank shells 122a-c, and small, medium, and large thigh shells 102a-c may be stacked with like components in a nested or stacked configuration. Additionally, as shown in FIG. 7, connected shank yokes 114a-c and shank shells 122a-c of different sizes (e.g., small, medium, or large) may be stacked with each other in a nested configuration. Likewise, connected thigh yokes 108a-c and thigh shells 102a-c of different sizes (e.g., small, medium, or large) may be stacked with each other in a nested configuration. The nesting capability of the modular orthosis system provides the added advantage of reduced shipping costs relative to shipping a traditional orthosis system.

Referring to FIG. 8, a second embodiment of the modular orthosis system 200 is shown. In the illustrated embodiment, the thigh shell connector 210 of the thigh yoke 208 includes a plurality of attachment points 246 that align with and correspond to attachment points 244 on the thigh shell 102, which enables the thigh shell 102 to be connected at any one of multiple locations along the thigh shell connector 210. This permits vertical adjustability of the thigh shell 102 with respect to the thigh shell connector 210. Although not shown in the illustrated embodiment, the shank yoke and shank shell may have the same configuration of attachment points for similar adjustability, which enables the shank shell to be connected at any one of multiple locations along the shank shell connector, allowing for vertical adjustability of the shank shell with respect to the shank shell connector.

Referring to FIGS. 9-11, a third embodiment of the modular orthosis system 300 is shown. In the illustrated embodiment, the thigh and shank yokes 308, 314 are joined to their respective thigh and shank shells 302, 310 via hinged connectors 350A, 350B, allowing the thigh and shank shells 302, 322 to pivot to match the contour of the patient’s leg more accurately in the sagittal plane, thereby distributing the forces against the leg more evenly and mitigating the risk of bruising or skin irritation, particularly along the upper edge 301 and lower edge 303 of the thigh shell, and along the upper edge 321 and lower edge 323 of the shank shell 322. In another embodiment, the thigh and shank yokes 308, 314 may be joined to their respective thigh and shank shells 302, 310 via a ball-and-socket connection.

In the illustrated embodiment, the modular orthosis system 300 includes connection pins 340 for attaching the thigh and shank yokes 308, 314 to their respective thigh and shank shells 302, 310. In an exemplary embodiment, the thigh and shank yokes 308, 314 may include embedded magnets 342 (visible in FIGS. 9 and 10) for drawing and retaining the connection pins 340. In either embodiment (i.e., with or without magnets), the connection pins 340 are preferably removable without the use of tools, improving the speed of donning and doffing. Reducing setup time is particularly important in a clinical setting where total time with the patient in a gait therapy session may be one hour or less.

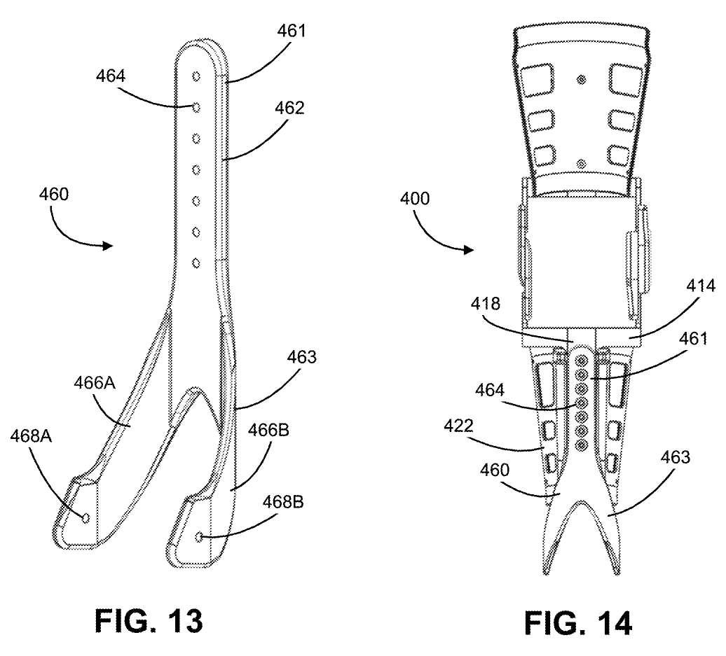

Referring to FIGS. 13-16, another embodiment of the modular orthosis system 400 may include an anti-slip element 460. As best shown in FIGS. 13 and 14, the anti-slip element 460 includes a shank connector 462 with mounting apertures 464 that allow an upper end 461 of the the anti-slip element 460 to be removably and adjustably secured to the shank shell connector 418 of the shank yoke 414, such that the anti-slip element 460 is positioned adjacent the rear exterior side of the shank shell 422. The mounting apertures 460 are adapted to align with corresponding apertures in shank shell connector 418 and allow the anti-slip element 460 to be secured at a desired vertical position along the shank shell connector 418 to accommodate different leg sizes (i.e., shorter or longer shank length) of different wearers. Thumb screws or any other suitable fasteners may be utilized to secure the anti-slip element 460 to the shank shell connector 418 of the shank yoke 414 via insertion through the mounting apertures 460 and corresponding apertures on the shank shell connector 418. In an alternate embodiment, the upper end 461 of the anti-slip element 460 may be secured to the shank shell 422 rather than the shank yoke 414. The lower end 463 of the anti-slip element 460 further includes a pair of wing portions 466A, 466B located beneath the shank connector 462. Each wing portion 466A, 466B may include a strap aperture 468A, 468B for securing a strap to anti-slip element 460.

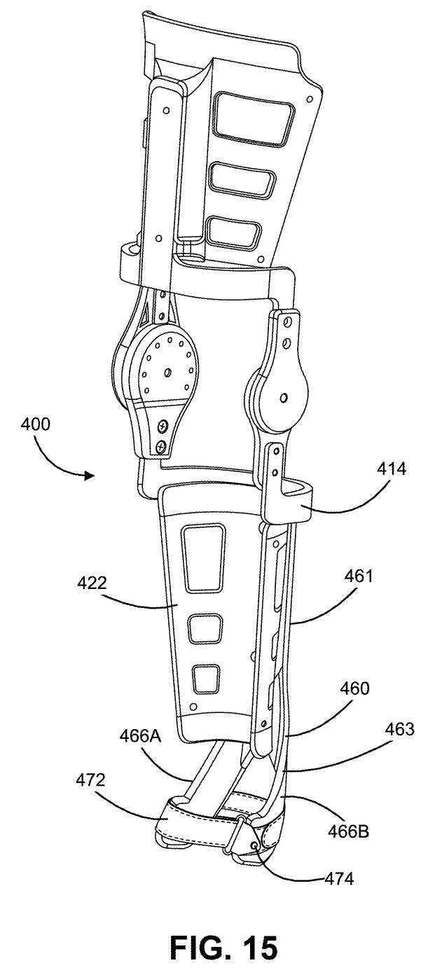

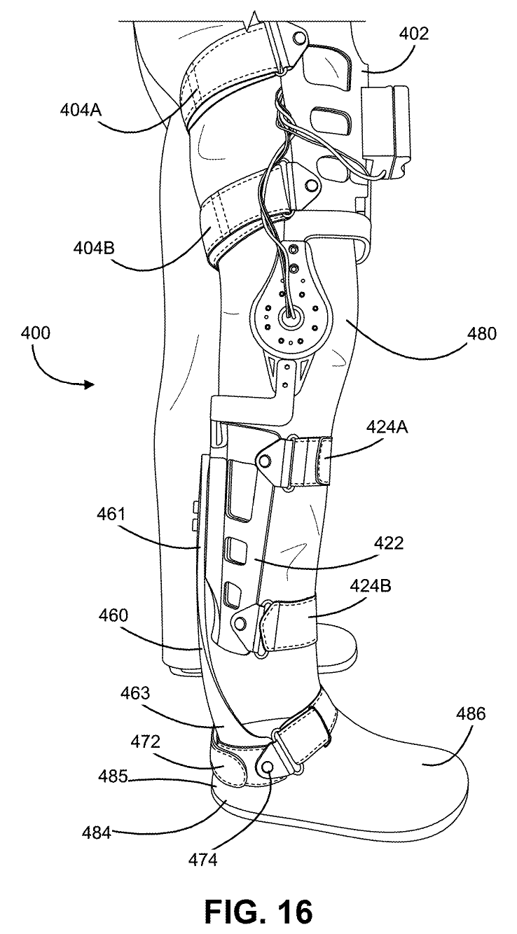

As best shown in FIGS. 15 and 16, the anti-slip element 460 is adapted to prevent the modular orthosis system 400 from sliding down the wearer’s leg 480. The anti-slip element 460 includes an adjustable anti-slip strap 472 that is secured, for example, to the strap apertures 468A, 468B of the wing portions 466A, 466B at the lower end 463 of the anti-slip element 460, via one or more fasteners 474, which may be rivets, screws, or any other suitable fasteners. The anti-slip strap 472 is adapted to wrap over the top of the wearer’s foot 486 and around the heel 484 and/or ankle 485. The anti-slip strap 472 may be adjustable in length and securable around the wearer’s foot 486 via any suitable fastening mechanism, such as hook and loop fasteners, for example. The anti-slip element 460 abuts the heel 484 of the wearer’s foot 486 to provide additional support to the modular orthosis system 400, in addition to the support provided by thigh shell straps 486A, 486B and the shank shell straps 488A, 488B. The anti-slip element 460 offloads some of the weight of the modular orthosis system 400 to the heel 484 of the wearer’s foot 486. In this way, the anti-slip element 460 is particularly effective at preventing slippage of the modular orthosis system 400 when the wearer also wears a separate existing ankle-foot orthosis in addition to the modular orthosis system 400.

While the principles of the invention have been described above in connection with preferred embodiments, it is to be clearly understood that this description is made only by way of example and not as a limitation of the scope of the invention.

1. A modular orthosis system, comprising:
a thigh yoke comprising a thigh shell connector, a first thigh yoke joint connector, a second thigh yoke joint connector, a thigh yoke link that connects the first thigh yoke joint connector to the second thigh yoke joint connector, the thigh yoke having a thigh yoke width extending from the first thigh yoke joint connector to the second thigh yoke joint connector, the thigh shell connector being rigidly connected to, and extending upwardly from, the thigh yoke link at a location between the first thigh yoke joint connector and the second thigh yoke joint connector;
a thigh shell adapted to be rigidly connected to the thigh shell connector, the thigh shell being adapted to engage a wearer’s thigh;
a shank yoke comprising a shank shell connector, a first shank yoke joint connector, a second shank yoke joint connector, a shank yoke link that connects the first shank yoke joint connector to the second shank yoke joint connector, the shank yoke having a shank yoke width extending from the first shank yoke joint connector to the second shank yoke joint connector, the shank shell connector being rigidly connected to, and extending downwardly from, the shank yoke link at a location between the first shank yoke joint connector and the second shank yoke joint connector; and
a shank shell adapted to be rigidly connected to the shank shell connector, the shank shell being adapted to engage a wearer’s shank;
wherein the first thigh yoke joint connector and the first shank yoke joint connector are each connected to a first pivoting joint and the second thigh yoke joint connector, and the second shank yoke joint connector are each connected to a second pivoting joint.

2. The modular orthosis system of claim 1, wherein the thigh shell connector is within a middle 30% of the thigh yoke width and the shank shell connector is within a middle 30% of the shank yoke width.

3. The modular orthosis system of claim 1, wherein the thigh shell connector is centered on the thigh yoke link between the first thigh yoke joint connector and the second thigh yoke joint connector and the shank shell connector is centered on the shank yoke link between the first shank yoke joint connector and the second shank yoke joint connector.

4. The modular orthosis system of claim 1, wherein the thigh shell and the shank shell are formed of a shell material and the thigh yoke and the shank yoke are formed of a yoke material, wherein the shell material has a lower flexural modulus than the yoke material.

5. The modular orthosis system of claim 1, further comprising:
an anti-slip element having an upper end and a lower end, the upper end adapted to be connected to the shank yoke, the lower end comprising a strap adapted to secure the lower end of the anti-slip element to a wearer’s ankle or foot;
wherein the anti-slip element is adapted to prevent the modular orthosis system from slipping downwardly along a wearer’s leg.

6. The modular orthosis system of claim 1, wherein the shell material is a polymeric material, and the yoke material is a metallic material.

7. The modular orthosis system of claim 1, wherein the first and second pivoting joints allow the thigh yoke and the shank yoke to pivot about a joint axis.

8. The modular orthosis system of claim 1, wherein the thigh shell includes at least one thigh strap adapted to secure the thigh shell to a human thigh and the shank shell includes at least one shank strap adapted to secure the shank shell to a human shank.

9. The modular orthosis system of claim 8, wherein thigh strap and the shank strap are adjustable.

10. The modular orthosis system of claim 1, wherein the thigh yoke link and the shank yoke link are each U-shaped.

11. The modular orthosis system of claim 1, wherein the first thigh yoke joint connector and the first shank yoke joint connector are each removably connected to the first pivoting joint and the second thigh yoke joint connector and the second shank yoke joint connector are removably connected to the second pivoting joint.

12. The modular orthosis system of claim 1, further comprising a thigh strain gauge located on the thigh shell connector.

13. A modular orthosis system, comprising:
at least one thigh yoke formed of a yoke material;
a plurality of thigh shells, each of the plurality of thigh shells being formed of a shell material and adapted to be removably, rigidly connected to the thigh yoke;
at least one shank yoke formed of the yoke material; and
a plurality of shank shells, each of the plurality of shank shells being formed of the shell material and adapted to be removably, rigidly connected to the shank yoke;
wherein the at least one thigh yoke is pivotally connectable to the at least one shank yoke by a first pivoting joint and a second pivoting joint, such that the at least one thigh yoke and at least one shank yoke are pivotal about a joint axis;
wherein the plurality of thigh shells includes a first thigh shell and a second thigh shell that is a different shape from the first thigh shell; and
wherein the plurality of shank shells includes a first shank shell and a second shank shell that is a different shape from the first shank shell.

14. The modular orthosis system of claim 13, wherein the second thigh shell is shaped and sized to accommodate a larger human thigh than the first thigh shell and the second shank shell is shaped and sized to accommodate a larger human shank than the first shank shell.

15. The modular orthosis system of claim 13, wherein the yoke material has a yoke flexural modulus, and the shell material has a shell flexural modulus that is less than the yoke flexural modulus.

16. The modular orthosis system of claim 13, wherein each of the plurality of thigh shells and the thigh shell connector are adapted to enable the thigh shell to be connected at any one of a plurality of locations along the at least one thigh shell connector.

17. The modular orthosis system of claim 13, wherein each of the plurality of shank shells and the shank shell connector are adapted to enable the shank shell to be connected at any one of a plurality of locations along the at least one shank shell connector.

18. The modular orthosis system of claim 13,
wherein at least one thigh yoke comprises a plurality of thigh yokes and the at least one shank yoke comprises a plurality of shank yokes;
wherein the plurality of thigh yokes includes a first thigh yoke and a second thigh yoke that is a different shape from the first thigh yoke; and
wherein the plurality of shank yokes includes a first shank yoke and a second shank yoke that is a different shape from the first shank yoke.

19. The modular orthosis system of claim 13,
wherein each of the at least one thigh yoke comprises a thigh shell connector, a first thigh yoke joint connector, a second thigh yoke joint connector, a thigh yoke link that connects the first thigh yoke joint connector to the second thigh yoke joint connector, the thigh yoke having the thigh yoke width extending from the first thigh yoke joint connector to the second thigh yoke joint connector, the thigh shell connector being rigidly connected to, and extending upwardly from, the thigh yoke link at a location between the first thigh yoke joint connector and the second thigh yoke joint connector; and
wherein each of the at least one shank yoke connector comprises a shank shell connector, a first shank yoke joint connector, a second shank yoke joint connector, a shank yoke link that connects the first shank yoke joint connector to the second shank yoke joint connector, the shank yoke having the shank yoke width extending from the first shank yoke joint connector to the second shank yoke joint connector, the shank shell connector being rigidly connected to, and extending downwardly from, the shank yoke link at a location between the first shank yoke joint connector and the second shank yoke joint connector.

20. The modular orthosis system of claim 19, wherein the thigh shell connector is within a middle 30% of the thigh yoke width and the shank shell connector is within a middle 30% of the shank yoke width.
